(19) United States
(12) Patent Application Publication
(10) Pub. No.: US 2017/0224225 A1
(43) Pub. Date: Aug. 10, 2017

(54) READING DEVICE

(71) Applicant: Chu-Yih Yu, Taipei (TW)

(72) Inventor: Chu-Yih Yu, Taipei (TW)

(21) Appl. No.: 15/015,140

(22) Filed: Feb. 4, 2016

(51) Int. Cl. A61B 5/01; A61B 5/00

(52) U.S. Cl. CPC A61B 5/01; A61B 5/0022; A61B 5/6833; A61B 5/4854

(57) ABSTRACT

A reading device, applied with at least one temperature sensing device and at least one external device; the temperature sensing device electrically connected with the reading device, comprising at least one temperature sensing patch which is capable of attaching on human skin, and sensing as well as recording body temperature change; the outer device being connected with, the reading device by a wireless communication; the reading device comprising: a housing, a data transmission port group, a screen, an external device connection port group, a memory unit, a processor unit, a control interface, and a wireless communication unit; the reading device is capable of reading and integrating the body temperature data obtained from the temperature sensing patch, and transmitting rapidly to the external device, whereby the medical personnel could quickly and easily know a patient's body temperature change, and effectively determine the patient's condition

### FIELD OF THE INVENTION

The present invention is relates to a reading device, particularly to a reading device capable of integrating temperature sensing data which is applied for medical assistance,

### BACKGROUND OF THE INVENTION

There is no two exactly the same green leaves, and there is no exactly the same gene between people, everyone taking the same medicine may not be effective, everyone's reactions to drugs are different, so in general, drug development from pharmaceutical companies or doctor's prescription, due to the technical limitations, can only treat the patient by “average level”, namely standardized medical care.

However, with the introduction of high-throughput sequencing technology and the development of large data processing capabilities, modern medical science is undergoing tremendous change from the traditional standardized medical care model to a personalized medicine paradigm, the personalized medicine that is “precision medicine” has become a new direction in modem medicine.

The purpose of the precision medicine is based on the patient's greatest wellbeing and the effective allocation of spending on socialized medicine, combined with modem epidemiology and preventive medicine, clinical diagnostics, therapeutics, molecular medicine, medical informatics technology, economics of health and medical sociology to let the traditional medicine move towards modernization and provide disease prevention, screening, diagnosis, treatment and rehabilitation program for everyone like tailored according to everyone's figure, with minimal resources to maximize health care, and improve the health of the overall population.

On the other hand, the Traditional Chinese Medicine (TCM) theory holds that the human body is viewed as a whole system that has nature operation and balance, the human will get sick if human body is out of balance, and the “different treatment according to the same sickness nature” already exist in the therapeutic strategy of the TCM that is corresponding to the “precision medicine” theory, That's because everyone's physiques are different, the same treatment will generate different reactions and results, it is necessary for each patient to be diagnosed by pathology and etiology, in order to make the most appropriate treatment

How can the precise medicine be combined with the TCM, combined the modern technology and traditional medical method with the scientific knowledge of the nature of the human body and disease to achieve the symmetry precision medicine, which is major problem.

In order to allow the TCM to achieve the precise medicine effect, it is very important to select the correct symmetric life information, which is a key marker of biology and physics; because the symmetric life information and the information gap [a state of balance] vary from person to person and from disease to disease, the medication and intervention are, based on these situations, the so-called symmetric balanced medical care that has direction in personalized medicine.

So, to select symmetric temperature change as a key marker is a preferable choice, which has five following reasons, 1th: humans are endotherins; 2nd: temperature is a statistics expression for the average kinetic energy of molecule; 3rd: it is a greatest common divisor for a variety of medicine; 4th: it is the most accurate, convenient, and cheapest way of life measuring way; 5th: it is symmetry and symmetry breaking theory based on physics frontier in the 21st century.

Although it has been approved that the symmetric temperature change can help to realize the symmetry precision medicine. Currently, the inventor has invented a temperature sensing patch that is used to measure body temperature and obtain the temperature sensing data.

Therefore, the present invention is to provide a reading device that is capable of integrating temperature sensing data obtained from the temperature sensing patch, thereby contributing to the precision medicine.

### SUMMARY OF INVENTION

The purpose of the invention is to provide a reading device applied with temperature sensing patch and integrated the body temperature data for contributing to the precision medicine.

For achieving the above-mentioned purpose, the technical scheme is a reading device (100), applied with at least one temperature sensing device (10) and at least one external device (20); the temperature sensing device (10) electrically connected with the reading device (100), comprising at least one temperature sensing patch (30) which is capable of attaching on human skin, and sensing as well as recording body temperature change; the outer device (20) being connected with the reading device by a wireless communication; the reading device comprising:

a housing (1), a data transmission port group (2) disposed at a top side surface area thereof, a screen (3) disposed on the top surface thereof, an external device connection port group (4), a memory unit (5), a processor unit (6) disposed within the housing (1), a control interface (7), and a wireless communication unit (8) installed inside the housing (1); wherein the data transmission port group (2) comprises a first port group (21) and a second port group (22) disposed correspondingly to the first port group (22) which are configured, to connect with the temperature sensing device (10); wherein the first and second port group (21, 22) are composed of ports (23) which are electrically connected with the temperature sensing patch (30);

wherein the external device connection port group (4) comprises a front port (41) configured to electrically connect with the external device (20) through a connection cord (43) and a rear port group (42) having a plurality of connecting ports (421) which are configured to connect with the temperature sensing devices (10) through connection cords (43); wherein the memory unit (5) is configured to store temperature sensing data (40) transmitted from the temperature sensing patch (30); wherein the processor unit (6) is configured to recharge the temperature sensing patch (30), process the temperature sensing data (40) and obtain consolidated information (50), and store the consolidated information (50) in the memory unit (5) when the temperature sensing patch (30) is plugged into the port (23) of the reading device (100); the external device (20) can receive the consolidated information (50) through connecting with the external device connection port group (4) or the wireless communication unit (8) of the reading device (100).

More preferably, wherein the control interface (7) comprises a circuit board (71) installed within the housing (1); a first button (72) disposed on a top surface of the housing (1) and configured to control the temperature sensing patch (30); and a second button (73) disposed on the top surface of the housing (1) and configured to control the communication unit (8).

More preferably, wherein the reading device (100) further comprises batteries (9) disposed within the housing (1); a battery container (11) corresponding to the batteries (9) and configured to receive the batteries (9); and a battery container cover (12) configured to close the battery container (11).

More preferably, wherein the communication unit (8) is one of the following or a combination of the following: infrared, Bluetooth, ZIGBEE, WIFI, WIMAX, 2G, 3G, 4G; wherein the external device (20) is one of the following: a smart phone, a tablet computer, a laptop computer, a desktop computer, a smart phone, a wearable device.

More preferably, wherein the housing (1) comprises an upper cover (13) provided for installing the screen (3) and the battery container (11); a lower cover (14) disposed correspondingly to the upper cover (13); and a containing case (15) disposed between the upper and lower cover (13, 14), and configured to contain the connection cords (43).

More preferably, wherein the port (23) is one of the following: Type-A USB port, Type-B USB port, Type-C USB port, Micro-A USB, Micro-B USB, Mini-A USB port, Mini-B USB port; wherein the front port (41) is one of the following: Type-A USB port, Type-B USB port, Type-C USB port, Micro-A USB, Micro-B USB, Mini-A USB port, Mini-B USB port; wherein the connecting ports (421) is one of the following: Type-A USB port, Type-B USB port, Type-C USB port, Micro-A USB, Micro-B USB, Mini-A USB port, Mini-B USB port.

More preferably, wherein the temperature sensing patch (30) comprises an outer housing (301), a circuit card (302), a temperature sensing unit (303) electrically connected with the circuit card (302), a battery (304), at least one port (305), a memory unit (306), and a processor chip (307); wherein the port (305) of the temperature sensing patch (30) is capable of connecting with the port (23) of the reading device (100); \A/herein the processor chip (307) is capable of automatically turning on the temperature sensing patch (30) and resetting the previous data in the memory unit (306) by a predetermined program, rather than through a manual switch, when the temperature sensing patch (30) is removed from the reading device (100).

More preferably, wherein the outer housing (301) comprises a receiving space (3011) defined therein; wherein the circuit card (302) is installed within, the a receiving space (3011); wherein the temperature sensing unit (303) electrically connected with the circuit card (302) defines a sensing terminal (3031) with one end thereof protruding over a surface, of the outer housing (301) to contact with human skin for sensing temperature; wherein the battery (304) is installed within the receiving space (3011) and is capable of providing the temperature sensing patch (30) with at least 24-hour staying power; wherein the port (305) is installed within the receiving space (3011), one end of which is located at a peripheral edge of the outer housing (301); wherein the memory unit (306) is installed on the circuit card (302) and is configured to store continuous digital data transmitted from the processor chip (307); and wherein the processor chip (307) is installed on the circuit card (302) and is capable of dealing with continuous digital data transmitted from the temperature sensing unit (303) within a predetermined time.

More preferably, wherein the temperature sensing patch (30) further comprises a light unit (308) electrically connected with the circuit card (302) and located within the received space (301 I) for a light indication of operation status of the temperature sensing patch (30); and an identification sticker (309) disposed on a top surface of one side of the outer housing (301) and configured to identify the temperature sensing patch (2).

More preferably, wherein the temperature sensing patch (30) further comprises an adhesive unit (A) disposed on another top surface of one side of the outer housing (304) and configured to attach on the human skin; the adhesive unit (A) having an adhesive layer (A1) and a release layer (A2) laminating with the adhesive layer (A1).

Compared to the prior art, the advantages of the present invention are:

Firstly, the reading device is capable of reading and integrating the body temperature data obtained from the temperature sensing patch, and transmitting rapidly to the external device, whereby the medical personnel could quickly and easily know a patient's body temperature change, and effectively determine the patient's condition. Secondly; the reading device can be connected with the external device by the connection cord or the wireless communication unit, thereby facilitating data transmission.

Thirdly, the design of the reading device is simple and its operation interface is easy to understand, so as to facilitate popularization and application.

### BRIEF DESCRIPTION OF THE DRAWINGS

FIG. 1 is a schematic view showing the reading device of the present invention applied with temperature sensing patches;

FIG. 2 is an exploded view of the present invention;

FIG. 3 is a top view of the present invention;

FIG. 4 is a rear view of the present invention;

FIG. 5 is a flow view showing the temperature sensing patches being removed from the reading device and being applied in a patient;

FIG. 6 is a flow view showing the temperature sensing patches being removed from the patient and being plugged in the reading device;

FIG. 7 is a schematic view showing body temperature data being transmitted from the reading device to an external device by wireless communication;

FIG. 8 is a schematic view of the temperature sensing patch; and

FIG. 9 is an exploded view of the temperature sensing patch.

### DETAILED DESCRIPTION OF THE PREFERRED EMBODIMENTS

The following description is a preferable embodiment according to the Figs.

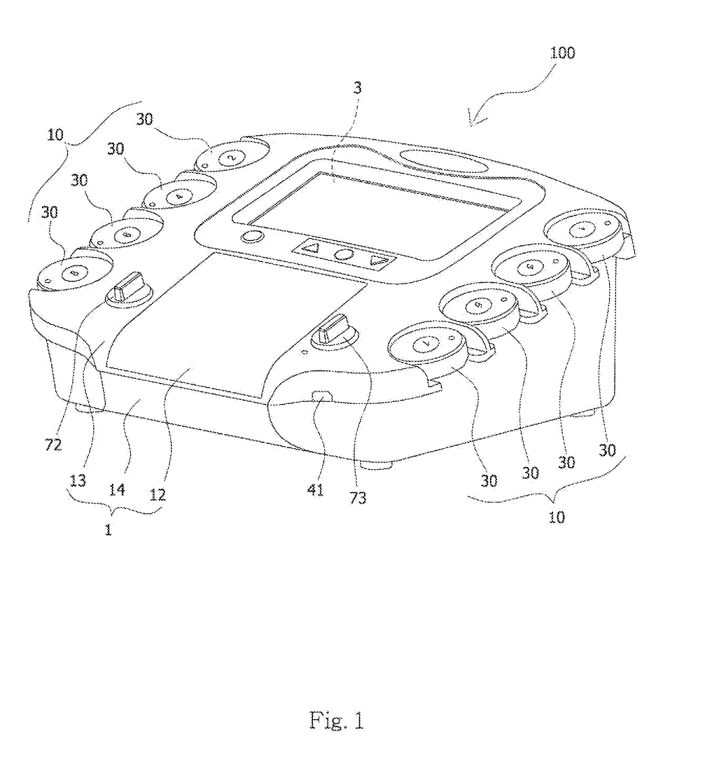

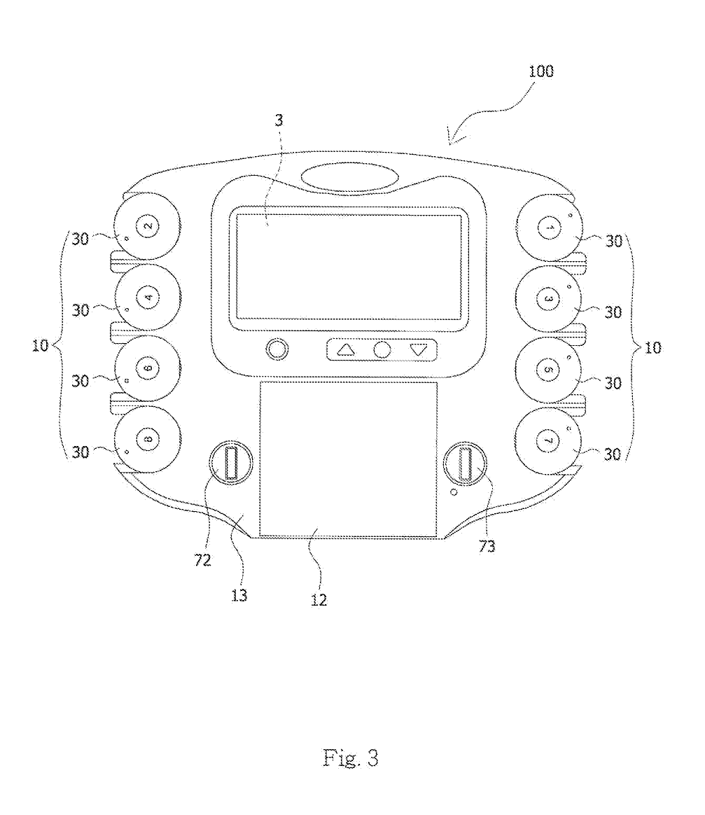

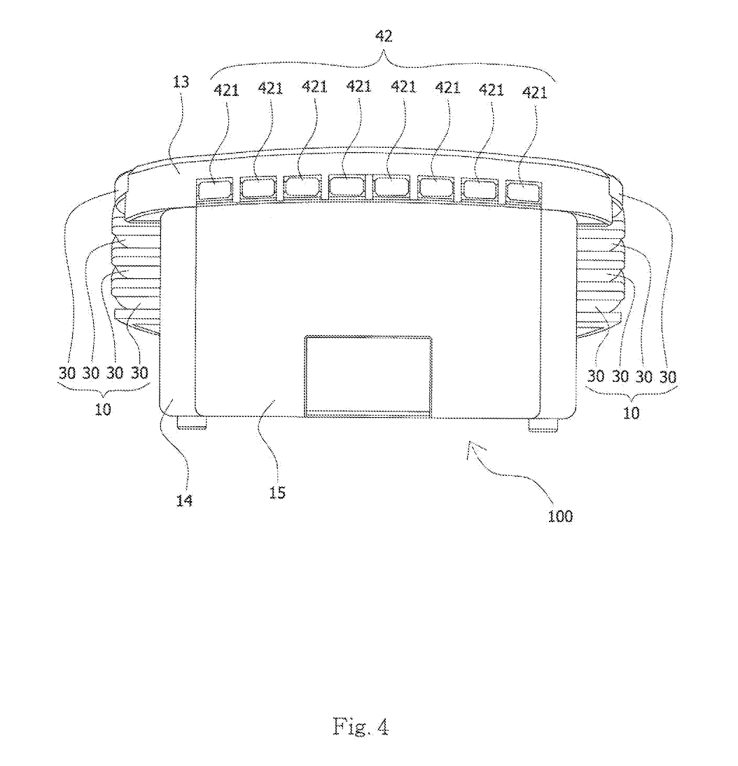

Referring to FIGS. 1-7, the Figures discloses a reading device (100) of temperature sensing data, applied with at least one temperature sensing device (10) and at least one external device (20); the temperature sensing device (10) electrically connected with the reading device (100), comprising at least one temperature sensing patch (30) which is capable of attaching on human skin, and sensing as well as recording body temperature change; the external device (20) being connected with the reading device by a wireless communication; the reading device comprising: a housing (1), a data transmission port group (2) disposed at a top side surface area thereof, a screen (3) disposed on the top surface thereof, an external device connection port group (4), a memory unit (5), a processor unit (6) disposed within the housing (1), a control unit (7), and a wireless communication unit (8) installed inside the housing (1);

wherein the data transmission port group (2) comprises a first port group (21) and a second port group (22) disposed correspondingly to the first port group (22), which are configured to connect with the temperature sensing device (10); wherein the first and second port group (21, 22) are composed of ports (23) which are electrically connected with the temperature sensing patch (30); wherein the external device connection port group (4) comprises a front port (41) configured to electrically connect with the external device (20) through a connection cord (43) and a rear port group (42) having a plurality of connecting ports (41) which are configured to connect with the temperature sensing devices (10) through connection cords (4); wherein the memory unit (5) is configured to store temperature sensing data (40) transmitted from the temperature sensing patch (30); wherein the processor unit (6) is configured to recharge the temperature sensing patch (30), process the temperature sensing data (40) and obtain consolidated information (50), and store the consolidated information (50) in the memory unit (5) when the temperature sensing patch (30) is plugged into the port (23) of the reading device (100); the external device (20) can receive the consolidated information (50) through the connecting with the external device connection port group (4) or the wireless communication unit (8) of the reading device (100).

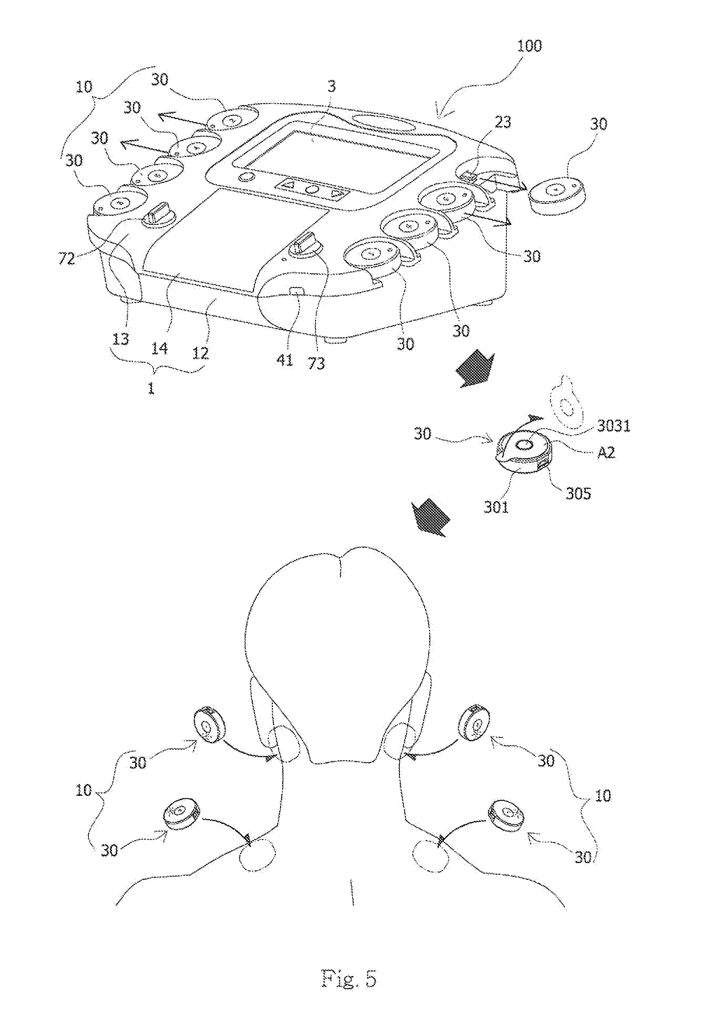

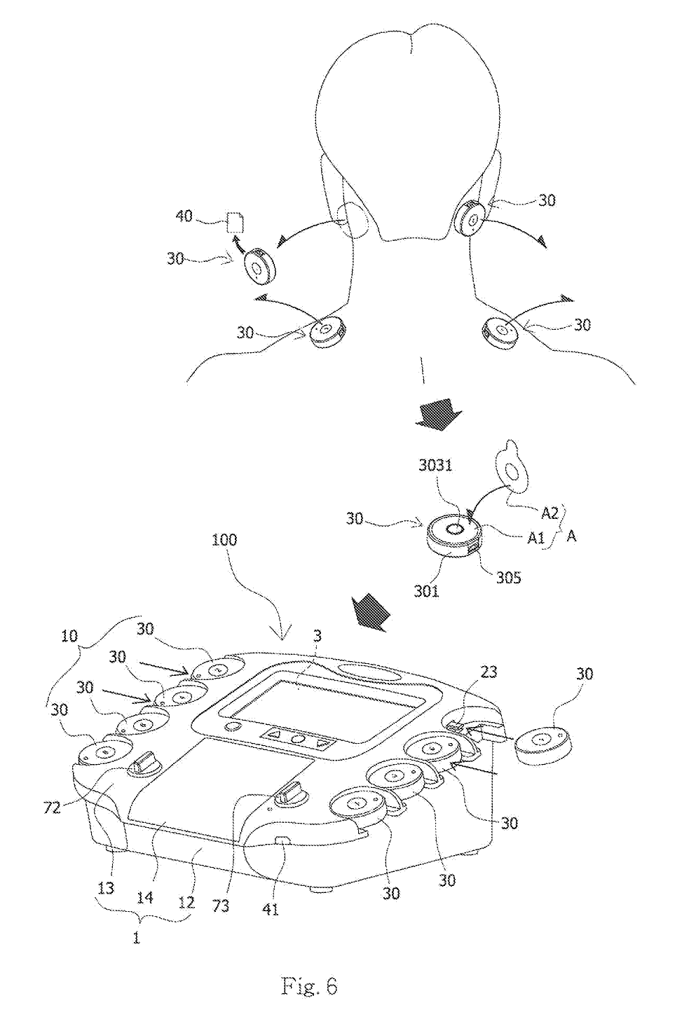

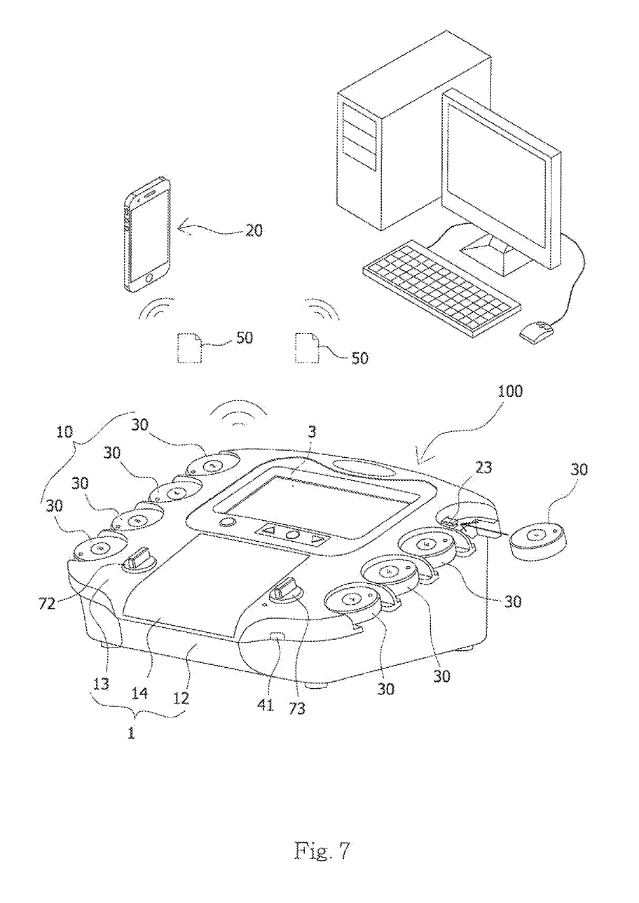

The reading device (100) is not only capable of recharging the temperature sensing patch (30) and reading the temperature sensing data (40) as shown in FIG. 5, but also capable of integrating the temperature sensing data as the consolidated information (50) and transmitting the consolidated information (50) to the external device (20) through the external device connection port group (4) or the wireless communication unit (8) as shown in FIGS. 6-7.

Besides, the consolidated information (50) is capable of being displayed as a curve chart that is easy to determine a location of lesion, the purpose of obtaining the consolidated information (50) is based on the traditional Chinese medicine theory which holds that the human body is viewed as a whole system that has nature operation and balance, the human will get sick if human body is out of balance, therefore the consolidated information (50) represents the balance change, the location of the out-of-balance can be determined by the curve chart, thereby facilitating diagnosing the pathology and etiology of the patient.

Moreover, the reading device (100) is installed with the wireless communication unit (8), which makes the data transmission more convenient.

Wherein the control unit (7) comprises a circuit board (71) installed within the housing (1); a first button (72) disposed on a top surface of the housing (1) and configured to control the temperature sensing patch (30); and a second button (73) disposed on the top surface of the housing (1) and configured to control the communication unit (8).

The first button (72) is configured to adjust the measuring time of the temperature sensing patch (30) for a specific predetermined time or real-time measurement.

The second button (73) is configured to control the open or close the wireless communication unit (8), thereby lowering the power consumption and extending more operation time.

Wherein the reading device (100) further comprises batteries (9) disposed within the housing (1); a battery container (11) corresponding to the batteries (9) and configured to receive the batteries (9); and a battery container cover (12) configured to close the battery container (11).

The reading device (100) can be powered by electrical power or the batteries (9); the batteries (9) can also recharge the temperature sensing patch (30).

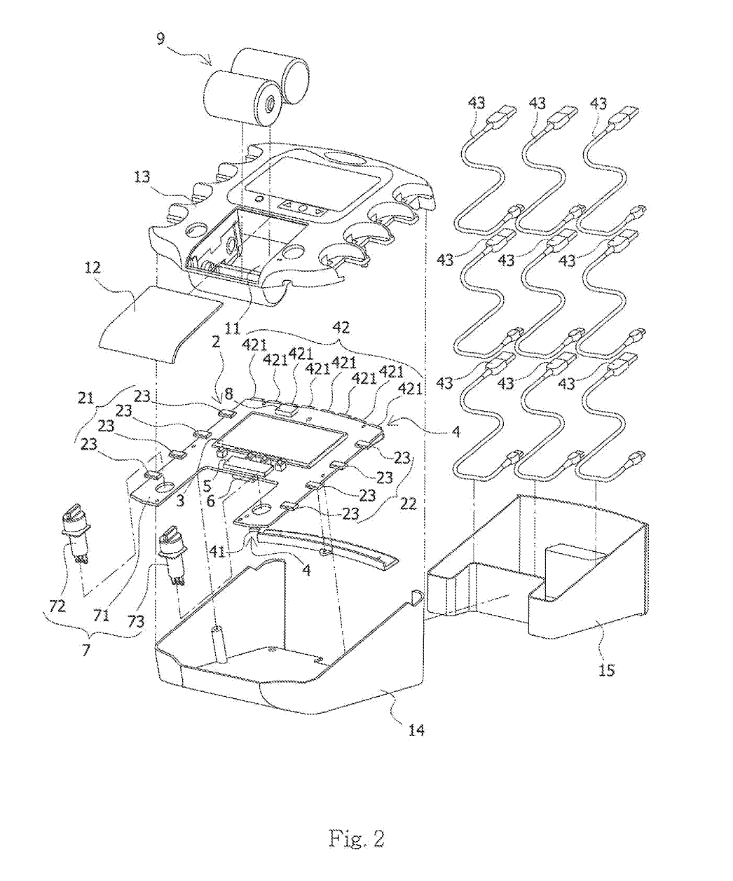

Referring to FIG. 2-4 again, wherein the communication unit (8) is one of the following or a combination of the following: infrared, Bluetooth, ZIGBEE, WIFI, WIMAX, 2G, 3G, 4G; wherein the external device (20) is one of the following: a smart phone, a tablet computer, a laptop computer, a desktop computer, a smart phone, a wearable device.

The communication unit (8) of the reading device (100) is capable of connecting with different external devices (20) in accordance with connection device installed therein, thereby facilitating data transmission.

Wherein the housing (1) comprises an upper cover (13) provided for installing the screen (3) and the battery container (11); a lower cover (14) disposed correspondingly to the upper cover (13); and a containing case (15) disposed between the upper and lower cover (13, 14), and configured to contain the connection cords (43).

The upper and lower cover (13, 14) is convenient to assemble the reading device (100) and the containing cover (14) is also convenient to carry the connection cords (43), thereby avoiding the connection cords (43) loss.

wherein the port (23) is one of the following: Type-A USB port, Type-B USB port, Type-C USB port, Micro-A USB, Micro-B USB, Mini-A USB port, Mini-B USB port; wherein the front port (41) is one of the following: Type-A USB port, Type-B USB port, Type-C USB port, Micro-A USB, Micro-B USB, USB port, Mini-B USB port; wherein the connecting ports (421) is one f the following: Type- A USB port, Type-B USB port, Type-C port Micro-A USB, Micro-B USB, Mini-A USB port Mini-B USB port.

The port (23), front port 41) and connecting port (421) of the reading device (100) are capable or being compatible with different external devices (20) ire accordance with connection device installed therein, thereby facilitating data transmission and battery recharge. The USB applied the reading device (100) not only can transmit the data and recharge the battery of the temperature sensing patch (30) at the same time, but also can reduce the size of the reading device (100).

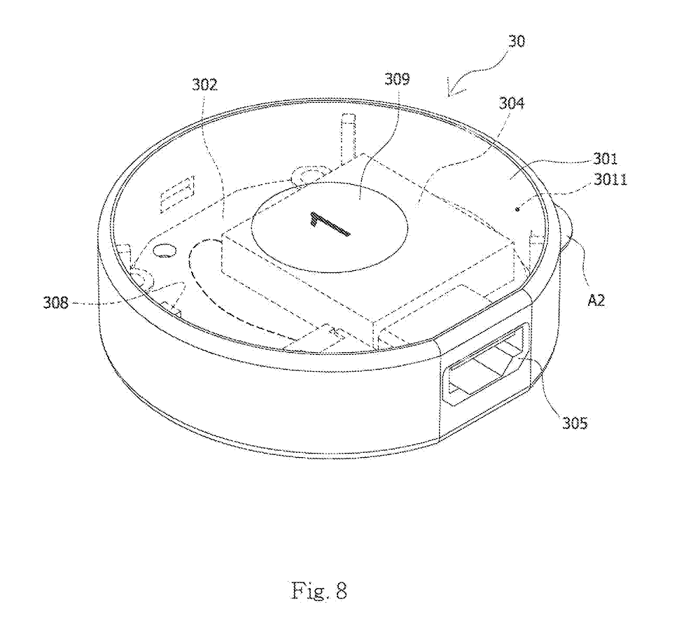

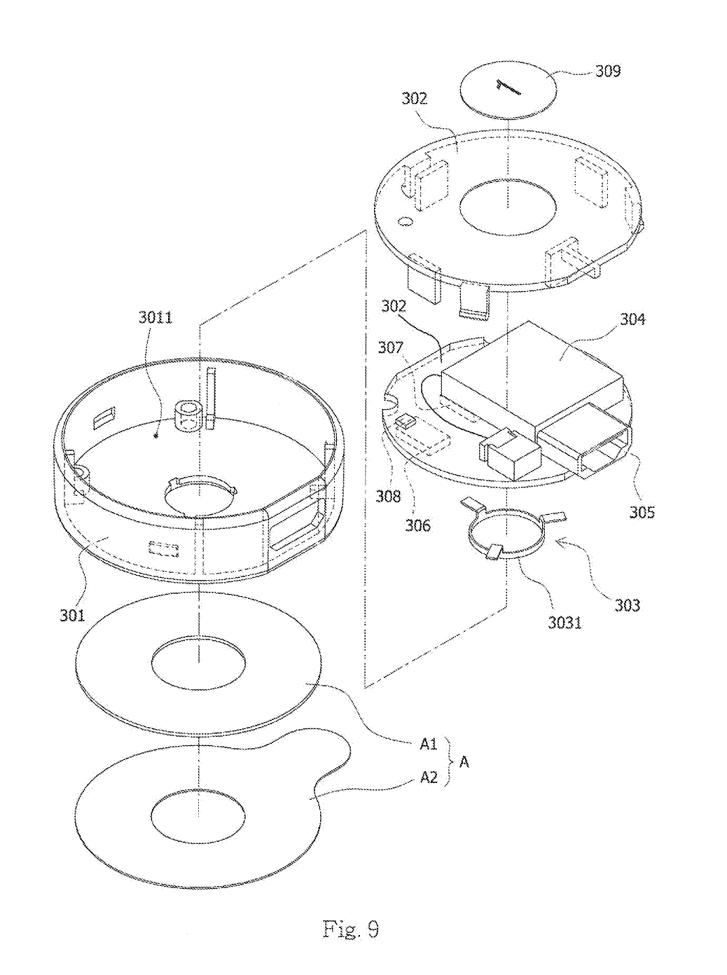

Referring to FIGS. 8-9, wherein the temperature sensing patch (30) comprises an outer housing (301), a circuit card (302), a temperature sensing unit (303) electrically connected with the circuit card (302), a battery (304), at least one port (305), a memory unit (306), and a processor chip (307); wherein the port (305) of the temperature sensing patch (30) is capable of connecting with the port (23) of the reading device (100); wherein the processor chip (307) is capable of automatically turning on the temperature se sing patch (30) and resetting the previous data in the memory unit (306) by a predetermined program, rather than through a manual switch, when the temperature sensing patch (30) is removed from the reading device (100).

When, in use, the temperature sensing patch (30) can be used by multi-point monitoring method to monitor the body temperature change for a long period time and provide the precise data, so the temperature sensing patch (30) is not as familiar as traditional thermometer does, it can provide the more practical reference data to the precision medicine.

Besides, there is no power switch in the temperature sensing patch (30), so that it can automatically turn on the power through the predetermined program when the temperature sensing patch (30) is removed from the reading device (100), and then the temperature sensing patch (30) will turn itself off when the battery runs out of power, so that the design can reduce user's burden on operation.

Moreover, the temperature sensing patch (30) can be synchronously used with multi-point measurement at the same time, thereby avoiding the data error generated by measuring in different time.

The design of the temperature sensing patch (30) is simple and small, so as to facilitate popularization and application.

Furthermore Except for controlling the switch of the temperature sensing patch (30) and handling digital information transmitted from the temperature sensing unit (303) within a predetermined time period, the processor chip (307) can also secure the data stored in the memory unit (306) before running out of the battery; the temperature sensing patch (30) is compatible with the reading device (100), which is capable of transmitting the data and recharging the battery (304) through the port (305).

Referring to FIGS. 8-9 again, wherein the outer housing (301) comprises a receiving space (3011) defined therein; wherein the circuit card (302) is installed within the a receiving space (3011); wherein the temperature sensing unit (303) electrically connected with the circuit card (302) defines a sensing terminal (3031) with one end thereof protruding over a surface of the outer housing (301) to contact with human skin for sensing temperature wherein the battery (304) is installed within the receiving space (3011) and is capable of providing the temperature sensing patch (30) with at least 24-hour staying power; wherein the port (305) is installed within the receiving space (3011), one end of which is located at a peripheral edge of the outer housing (301); wherein the memory unit (306) is installed on the circuit card (302) and is configured to store continuous digital data transmitted from the processor chip (307); and wherein the processor chip (307) is installed on the circuit card (302) and is capable of dealing with continuous digital data transmitted from the temperature sensing unit (303) within a predetermined time.

At least the 24-hour staying power of battery capacity for the temperature sensing patch (30) can fully gain more temperature data from human body based on the a person's daily activity.

wherein the temperature sensing patch (30) further comprises a light unit (308) electrically connected with the circuit card (302) and located within the received space (3011) for a light indication of operation status of the temperature sensing patch (30); and an identification sticker (309) disposed on a top surface of one side of the outer housing (301) and configured to identify the temperature sensing patch (2).

there is no manual power switch installed on the temperature sensing patch (30), so the light unit (308) would automatically light up to identify the battery power when in use, if the battery (304) is running out of power, the light unit (308) will be changed to other color and/or twinkling light to alert users,

wherein the temperature sensing patch (30) further comprises an adhesive unit (A) disposed on another top surface of one side of the outer housing (301) and configured to attach on the human skin.; the adhesive unit (A) having an adhesive layer (A1) and a release layer (A2) laminating with the adhesive layer (A1).

The temperature sensing patch (30) can be directly attached on the human's skin after removed from the release layer. Besides, for complying with the medical standards, the adhesive unit (A) can be reusable or disposable.

It will be evident to those skilled in the art that the invention is not limited to the details of the foregoing illustrated embodiments and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

1. A reading device (100), applied with at least one temperature sensing, device (10) and at least one external device (20); the temperature sensing device (10) electrically connected with the reading device (100), comprising at least one temperature sensing patch (30) which is capable of attaching on human skin, and sensing as well as recording body temperature change; the outer device (0) being connected with the reading device by a wireless communication; the reading device comprising:
a housing (1), a data transmission port group (2) disposed at a top side surface area thereof, a screen (3) disposed en the top surface thereof, an external device connection port group (4), a memory unit (5), a processor unit (6) disposed within the housing (1), a control interface (7), and a wireless communication unit (8) installed inside the housing (1);
wherein the data transmission port group (2) comprises a first port group (21) and a second port group (22) disposed correspondingly to the first port group (22), which arc configured to connect with the temperature sensing device (10); wherein the first and second port group (21, 22) are composed of ports (23) which are electrically connected wits the temperature sensing patch (30);
wherein the external device connection,port group (4) comprises a front port (41) configured to electrically connect with the external device (20) through a connection cord (43) and a rear port group (42) having a plurality of connecting ports (421) which are configured to connect with the temperature sensing devices (10) through connection cords (43);
wherein the memory unit (5) is configured to store temperature sensing data (40) transmitted from the temperature sensing patch (30);
wherein the processor unit (6) is configured to recharge the temperature sensing patch (30), process the temperature sensing data (40) and obtain consolidated information (50), and store the consolidated information (50) in the memory unit (5) when the temperature sensing patch (30) is plugged into the port (23) of the reading device (100); the external device (20) can receive the consolidated information (50) through connecting with the external device connection port group (4) or the wireless communication unit (8) of the reading device (100).

2. The reading device according to claim 1, wherein the control interface (7) comprises a circuit board (71) installed within the housing (1); a first button (72) disposed on a top surface of the housing (1) and configured to control the temperature sensing patch (30); and a second button (73) disposed on the top surface of the housing (1) and configured to control the communication unit (8).

3. The reading device according to claim 1, wherein the reading device (100) further comprises batteries (9) disposed within the housing (1); a battery container (11) corresponding to the batteries (9) and configured to receive the batteries (9); and a battery container cover (₁₂) configured to close the battery container (11).

4. The reading device according to claim 1, wherein the communication unit (8) is one of the following or a combination of the following: infrared, Bluetooth, ZIGBEE, WIFI, WIMAX, 2G, 3G, 4G;
wherein the external device (20) is one of the following: a smart phone, a tablet computer, a laptop computer, a desktop computer, a smart phone, a wearable device.

5. The reading device according to claim 1, wherein the housing (1) comprises an upper cower (13) provided for installing the screen (3) and the battery container (11); a lower cover (14) disposed correspondingly to the upper cover (13); and a containing case (15) disposed between the upper and lower cover (13, 14), and configured to contain the connection cords (43).

6. The reading device according to claim 1, wherein the port (23) is one of the following: Type-A USB port, Type-B USB port, Type-C USB port, Micro-A USB Micro-B USB, Mini-A USB port, Mini-B USB port;
wherein the front port (41) is one of the following: Type-A USB port, Type-B USB port, Type-C USB port, Micro-A USB, Micro-B USB, Mini-A USB port, Mini-B USB port;
wherein the connecting ports (421) is one of the following: Type-A USB port, Type-B USB port, Type-C USB port, Micro-A USB, Micro-B USB Mini-A USB port, Mini-B USB port.

7. The reading device according to claim 1, wherein the temperature sing patch (30) comprises an outer housing (301), a circuit card (302), a temperature sensing unit (303) electrically connected with the circuit card (302), a battery (304), at least one port (305), a memory unit (306), and a processor chip (307);
wherein the port (305) of the temperature sensing patch (30) is capable of connecting with the port (23) of the reading device (100);
wherein the processor chip (307) is capable of automatically turning on the temperature sensing patch and resetting the previous data in the memory unit (306) by a predetermined program, rather than through a manual switch, when the temperature sensing patch (30) is removed from the reading device (100),

8. The reading device according to claim 7, wherein the outer housing (301) comprises a receiving space (3011) defined therein;
wherein the circuit card (302) is installed within the a receiving space (3011);
wherein the temperature sensing, unit (303) electrically connected with the circuit card (302) defines a sensing terminal (3031) with one end thereof protruding over a surface of the outer housing (301) to contact with human skin for sensing temperature;
wherein the battery (304) is installed within the receiving space (3011) and is capable of providing the temperature sensing patch (30) with at least 24-hour staying power;
wherein the port (305) is installed within the receiving space (3011), one end of which is located at a peripheral edge or the outer housing (301);
wherein the memory unit (306) is installed on the circuit card (302) and is configured to store continuous digital data transmitted from the processor chip (307); and
wherein the processor chip (307) is installed on the circuit card (302) and is capable of dealing with continuous digital data transmitted from the temperature sensing unit (303) within a predetermined time.

9. The reading device according to claim 7, wherein the temperature sensing patch (30) further comprises a light unit (308) electrically connected with the circuit card (302) and located within the received space (3011) for a light indication of operation status of the temperature sensing patch (30); and an identification sticker (389) disposed on a top surface of one side of the outer housing (301) and configured to identify the temperature sensing patch (2).

10. The reading device according to claim 7, wherein the temperature sensing patch (30) further comprises an adhesive unit (A) disposed on another top surface of one side of the outer housing (301) and configured to attach on the human skin; the adhesive unit (A) having an adhesive layer (A1) and a release layer (A2) laminating with the adhesive layer (A1).
